(19) United States
(12) Patent Application Publication
(10) Pub. No.: US 2010/0241028 A1
(43) Pub. Date: Sep. 23, 2010

(54) Navigable Tissue Treatment Tools

(75) Inventors: Jay Johnson, Maple Plain, MN (US); Tom Borillo, Medina, MN (US); Paige Hastings, Bloomington, MN (US); David J. McKinley, Chanhassen, MN (US)

Correspondence Address: INSKEEP INTELLECTUAL PROPERTY GROUP, INC, 2281 W. 190TH STREET, SUITE 200, TORRANCE, CA, 90504, US

(73) Assignee: superDimension, Ltd.

(21) Appl. No.: 12/643,917

(22) Filed: Dec. 21, 2009

Related U.S. Application Data
(60) Provisional application No. 61/139,501, filed on Dec. 19, 2008.

(51) Int. Cl. A61B 10/04; A61B 19/00; A61M 36/12

(52) U.S. Cl. 600/567; 606/130; 600/569; 600/7

(57) ABSTRACT

Tools for accessing tissue are described for use with a locatable guide of a navigation system. In preferred embodiments, said tools are attachable to a distal tip of a locatable guide, such that the location of the tool, preferably in six degrees of freedom, is known while the tool is being used.

### RELATED APPLICATIONS

This application claims priority to U.S. Provisional Application Ser. No. 61/139,501 filed Dec. 19, 2008 entitled Biopsy Tools, which is hereby incorporated herein by reference.

### BACKGROUND OF THE INVENTION

Identifying and treating lung tissue abnormalities presents challenges that are somewhat unique to the lungs. If a tissue lesion or tumor is to be identified and excised surgically, the chest wall must be opened to provide access to the lungs. Opening the chest wall is a common procedure but one that presents risks of infection and lengthy recovery time, nonetheless. If a tissue lesion or tumor is to be identified endoscopically, the complicated bronchial maze must be navigated.

Technology has been developed that allows a physician to track, in real-time, the position of a probe (hereinafter “locatable guide” or “LG”) traveling through the airways. This technology incorporates a plurality of coils at the end of an LG and a magnetic field generator outside of the patient. The patient is placed in the magnetic field created by the generator. As the LG is navigated through the airways, electrical current is induced in the coils and sent via conductors to a computer. The computer can calculate the position and orientation of the probe based on the relative strengths of the current being induced. This technology is shown and described in greater detail in U.S. Pat. Nos. 7,233,820 6,226,543, 6,188,355, 6,380,732, 6,593,884, 6,711,429, 6,558,333, 6,887,236, 6,615,155, 6,574,498, 6,947,788, 6,996,430, 6,702,780, and 6,833,814; and U.S. Patent Publications 20050171508, 20030074011, 20020193686, each of which is incorporated by reference herein in its entirety and also PCT application WO 03/086498 titled “Endoscope Structure and Techniques for Navigation in Branched Structure” to Gilboa, fully incorporated herein by reference.

One type of tool used with the above-described system is a biopsy tool. Biopsy tools are designed to remove and retrieve small tissue samples from a suspected lesion for analysis and identification in a laboratory. Several factors are considered when taking biopsies of tissue such as biopsy location, biopsy size, and the number of samples needed from a suspected lesion.

The above system and apparatus are aimed at the first consideration, target location, and provide a system that enables a physician to navigate standard bronchoscopic tools, such as biopsy tools, to a target located in the lung. In its basic operation, the target is first identified in the CT data, and then the locatable guide is navigated to the target. The locatable guide is then removed from a sheath surrounding the LG and the sheath is then used as an extended working channel (“EWC”) through which a biopsy tool may be passed to the target location.

Once the LG is removed, however, the locating system is no longer useful in identifying the location of the tip of the EWC. Hence, with regard to the biopsy tool, the physician is effectively “operating in the blind.” Additionally, quite often it is desired to take multiple samples from various locations within a lesion so as to ensure a representative sampling of tissue.

It would be therefore advantageous to develop biopsy tools that have improved capabilities that address these identified needs. Namely, it would be advantageous to develop a biopsy, or tissue treatment tool that retrieves biopsy samples from or otherwise treats tissue in a variety of locations within a lesion simultaneously. It would also be advantageous to develop a biopsy tool that retrieves an adequate tissue sampling without requiring the removal of the LG from the EWC.

### SUMMARY OF THE INVENTION

The present invention represents a step forward in endoscopic biopsy and treatment procedures by providing tools designed for use with a three-dimensional locating system. The tools of the present invention are constructed to obviate the need for multiple navigation steps when taking one or more samples from a biopsy site, or performing one or more treatment procedures.

One aspect of the present invention provides a tool that attaches to the end of an LG. Such a device provides many advantages. First, a small, attachable tool presents a cost savings when compared to a more traditional tool long enough to be routed through a catheter. Typically, LGs and biopsy tools are single-use disposable devices. Reducing the material and complexity of a disposable is always preferred in order to reduce costs. Second, because the attachable tool is attached to the LG, the LG maintains its functionality and, therefore, can be used to monitor the location of the tool throughout a procedure. Moreover, the locatable tool may be used to take samples from or treat tissue in multiple locations in a lesion without retracting the tool and repositioning the EWC. Also, because the tool is not removed from the lungs between samples, a single tool may be used. Third, input from the LG may be used to map and display the places in the lesion where samples have been taken.

Another aspect of the present invention provides a biopsy tool that includes multiple extensions that radiate from its distal tip when deployed. These extensions are designed to take individual samples from multiple locations in a target site simultaneously. This device may be used in the EWC after the LG is removed, as the multiple extensions effectively biopsy an entire lesion simultaneously, thereby reducing the need for location and tracking during the biopsy. Alternatively, this “flowering” biopsy tool may be incorporated into an LG, such that the LG does not need to be removed from the EWC prior to deployment.

### BRIEF DESCRIPTION OF THE DRAWINGS

FIG. 1 is a perspective view of an embodiment of a device of the present invention;

FIG. 2 is a perspective view of another embodiment of a device of the present invention;

FIG. 3 is a perspective view of an embodiment of a device of the present invention;

FIG. 4 is a perspective view of an embodiment of a device of the present invention; and,

FIG. 5 is a perspective view of an embodiment of a device of the present invention.

### DETAILED DESCRIPTION OF THE INVENTION

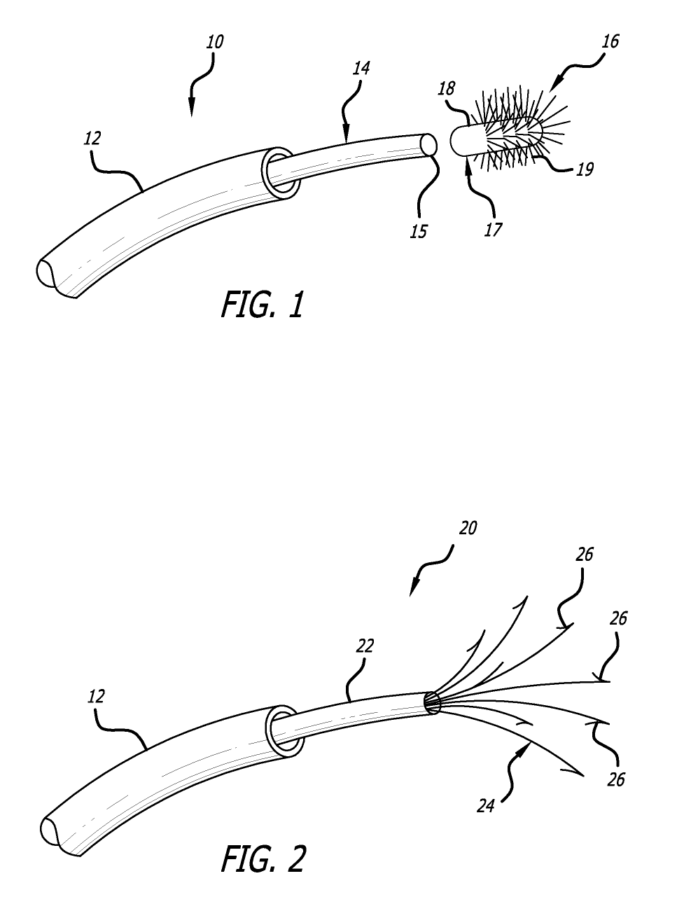

Referring now to FIG. 1, there is shown an embodiment of a device 10 of the present invention. The device 10 generally comprises a delivery catheter or EWC 12, an LG 14, and an attachable tool 16. The tool 16 is shown as a brush, but may be take any form of any device. In this sense, FIG. 1 is showing the general concept of an attachable tool 16, as well as providing a specific embodiment of a tool, in this case, a biopsy brush. Addressing the specific embodiment of a brush, the bristles of brush 16 may be configured with barbs or other tissue snagging devices if it is desired to increase the amount of tissue retrieved by the device 10.

Referring to the general embodiment of a generic tool, tool 16 has a proximal attachment portion 17 that is attachable to, or configured to mate with, the distal end 15 of the LG 14 by an end user, such as a physician or physician's assistant. In preferred embodiments, the attachment portion 17 includes a proximal skirt 18 that is sized to fit over the distal end 15 of the LG 14. If it is desired to create a permanent attachment between the tool 16 and the LG 14, an appropriate adhesive may be used to affix the tool 16 to the LG 14. Alternatively, the distal end 15 of the LG may be configured to removably mate with the tool 16. For example, distal end 15 may include a snap connector, a threaded connector, a luer lock, etc. Similarly, the skirt 18 may be configured appropriately to accept whatever configuration is formed on the distal tip 15 of the LG 14. As shown, the distal tip 15 serves as a male component while the skirt 18 serves as a female component. One skilled in the art will understand that the distal tip 15 of the LG 14 may be configured as a female component for accepting a corresponding male component of the biopsy tool 16.

In a preferred embodiment, the LG 14 is a standard LG and the tool 16 is available in a variety of forms. Hence, a physician is able to attach any of several tools 16 to the distal end 15 of a standard LG 14 using either a permanent or removable adhesive. Each of the various embodiments of tools described herein generally include a tool portion and an attachment portion. In the example shown in FIG. 1, the tool 16 has an attachment portion 17, described above as a proximal skirt 18, and a tool portion 19, in this case a biopsy brush.

Another preferred embodiment of a device 20 is shown in FIG. 2. Like the device 10 of FIG. 1, the device 20 is deliverable through a catheter or EWC 12. The device 20 further includes a biopsy tool 24 that radiates out of a sheath 22 when the tool 24 is advanced through the sheath 22. This biopsy tool 24 allows several samples to be taken simultaneously, covering a relatively large area. The biopsy tool 24 includes a plurality of tines 26, each of which is shown in the figure as being equipped with a barb for catching tissue.

It is envisioned that the sheath 22 may comprise an LG that has been modified to include a lumen for accommodating the biopsy tool 24. The device 20 is designed such that, after the tissue samples are taken, the biopsy tool is partially retracted into the sheath 22 until the tines 24 are brought together. The tines 24 and the sheath 22 may then be retracted into the EWC 12. Leaving the tines 24 partially extended prevents the loss of the tissue samples taken.

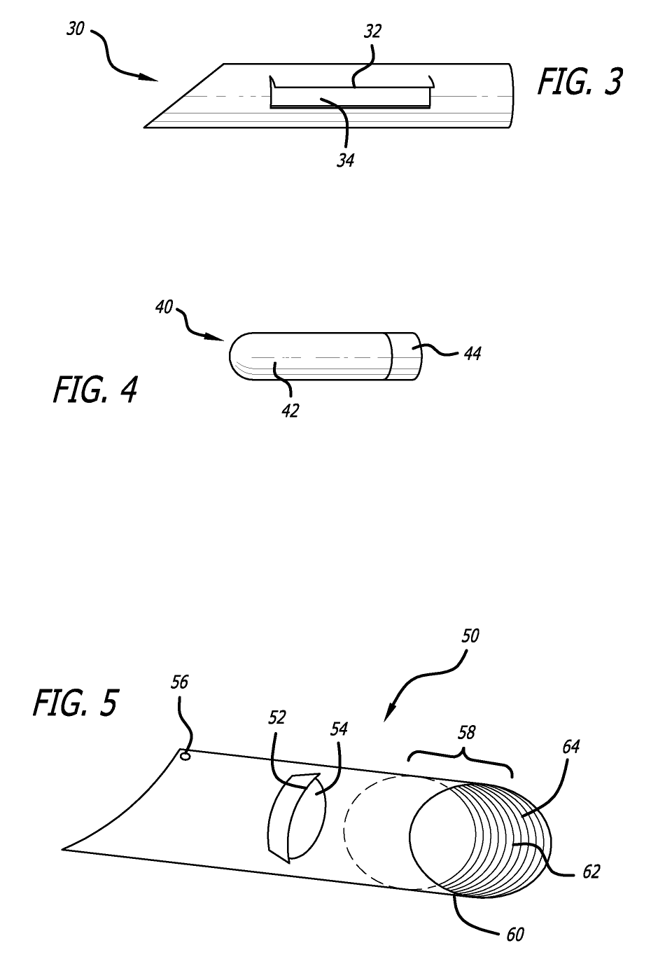

FIGS. 3-5 show various embodiments of tools according to the present invention. It is understood that all of these tools are attachable to the distal end of an LG in any of the various manners described above.

Referring first to FIG. 3, there is shown a biopsy needle 30 according to the present invention. Biopsy needle 30 includes a scalloping blade 32 on a side surface of the needle 30. The scalloping blade allows a relatively large sample of tissue to be excised without the need for a jaw mechanism. The needle 30 also includes a hollow interior cavity 34 for receiving the tissue sample. In operation, the needle 30 is advanced into the target tissue and rotated. During rotation, the scallop blade 32 cuts a tissue sample and directs the sample into the cavity 34. Upon retraction, any remaining connection to the tissue is severed by a distal edge of the blade 32. Advantageously, little damage is done to any tissue that may lie between the targeted area, and the body lumen through which the LG was navigated to the site. Similarly, none of this tissue is sampled, as may be the case when using a brush device. Because the needle is rigid, the needle itself may easily be displayed using the navigation system, as the spatial relationship between the needle and the LG sensor is fixed.

FIG. 4 shows a seed implantation tool 40, including a seed 42 and a detachable coupling 44. The detachable coupling allows the marker seed 42 to be detached from the distal end of the LG once the marker seed 42 is inserted into a target location. The detachable coupling may be electrolytic, dissolvable, meltable, threaded, shaped-memory metal, stress-induced martensite, or any other known detachment mechanism used in percutaneously-delivered devices. The seed 42 may be a marker seed or a therapeutic seed. Many seeds acceptable for this purpose are shown and described in U.S. Publication 2009/0240140 entitled “Target Identification Tool for Intra-Body Localization” the entirety of which is incorporated by reference herein. Non-limiting examples include seeds adapted for use in marking locations, locatable visually or using ultrasound, Geiger meters, radio receivers, fluoroscopes, etc; or therapeutic seeds designed to administer drugs, chemotherapy, radiation therapy, cryo-therapy, ablation energy, or the like.

FIG. 5 shows a biopsy needle 50 according to the present invention. Biopsy needle 50 includes a scalloping blade 52 on a side surface of the needle 50, oriented so the blade faces a proximal direction. The scalloping blade allows a relatively large sample of tissue to be excised without the need for a jaw mechanism. The needle 50 also includes a hollow interior cavity 54 for receiving the tissue sample. In operation, the needle 50 is advanced into the target tissue and retracted. During retraction, the scallop blade 52 cuts a tissue sample and directs the sample into the cavity 54. An air escape port 56 near a distal end of the needle 50 allows air or fluid to escape from the cavity 54, to more easily allow tissue to fill the cavity. Because the needle is rigid, the needle itself may easily be displayed using the navigation system, as the spatial relationship between the needle and the LG sensor is fixed. FIG. 5 is oriented to show the attachment portion 58. It is understood that the attachment portion 58 of FIG. 5 is representative of an attachment portion usable with any of the embodiments described herein. The attachment portion 58 includes a skirt 60 defining a female receptacle 62. The skirt 60, in this embodiment is shown with an interior surface containing threads 64. As state above, the threads may be replaced with any appropriate fastening device, including, but not limited to, luer lock, snap fit, friction fit, etc., and may be supplemented with an adhesive. It is also envisioned that the device 50 of the present invention may be designed to take tissue samples at the distal end of the device. As shown, the device includes a distal tip shaped like a scoop. An opening (not shown) to the cavity 54 could be provided. In this case, the scallop blade 52 would be omitted and the air escape port 56 would be located in a proximal location.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

We claim:

1. An endoscopic tool attachable to a locatable guide, the tool comprising:
a distal tool portion;
an attachment portion proximal of the tool portion;
wherein said attachment portion is configured to mate with a distal tip of a locatable guide.

2. The endoscopic tool of claim 1 wherein said attachment portion comprises a skirt defining an opening into which a distal tip of a locatable guide may be inserted.

3. The endoscopic tool of claim 2 wherein said skirt comprises an inner surface defining threads configured to mate with threads formed on a distal tip of a locatable guide.

4. The endoscopic tool of claim 1 wherein said distal tool portion comprises a biopsy brush.

5. The endoscopic tool of claim 1 wherein said distal tool portion comprises biopsy needle.

6. The endoscopic tool of claim 5 wherein said biopsy needle comprises a pointed distal end and a scallop blade extending from a side surface of said needle, said scallop blade usable to excise tissue and direct excised tissue into a an opening that leads to an inner cavity of said biopsy needle.

7. The endoscopic tool of claim 6 wherein said scallop blade extends in a proximal direction.

8. The endoscopic tool of claim 6 wherein said scallop blade extends in a radial direction.

9. A method of accessing tissue comprising;
providing a locatable guide including a location sensor usable to provide navigational information to a user advancing said locatable guide to targeted tissue within a body lumen;
attaching an endoscopic tool to a distal end of said locatable guide;
navigating said locatable guide to said targeted tissue;
performing a procedure on said tissue using said endoscopic tool.

10. The method of claim 9 wherein said step of attaching an endoscopic tool to a distal end of said locatable guide comprises attaching an endoscopic tool having:
a distal tool portion;
an attachment portion proximal of the tool portion;
wherein said attachment portion is configured to mate with a distal tip of a locatable guide.

11. The method of claim 10 wherein said step of attaching an endoscopic tool to a distal end of said locatable guide comprises attaching an endoscopic tool further having an attachment portion that comprises a skirt defining an opening into which a distal tip of a locatable guide may be inserted.

12. The method of claim 9 wherein performing a procedure on said tissue using said endoscopic tool comprises performing a biopsy on said tissue.

13. The method of claim 9 wherein performing a procedure on said tissue using said endoscopic tool comprises implanting a seed into said tissue.

14. The method of claim 9 wherein attaching an endoscopic tool to a distal end of said locatable guide comprises attaching a biopsy needle to a distal end of said locatable guide.

15. The method of claim 9 wherein attaching an endoscopic tool to a distal end of said locatable guide comprises attaching a biopsy brush to a distal end of said locatable guide.

16. The method of claim 9 wherein attaching an endoscopic tool to a distal end of said locatable guide comprises attaching a seed implantation device to a distal end of said locatable guide.

17. A probe deliverable through a working channel of a bronchoscope to biopsy tissue, said probe comprising:
a sheath defining a lumen;
a plurality of tines, at least some of which including barbs;
wherein said tines are advanceable out of a distal end of said sheath and configured such that, when advanced, said tines radiate outwardly to access tissue.

18. The probe of claim 17 wherein said tines are further retractable back into said sheath.
